(19) United States
(12) Patent Application Publication
(10) Pub. No.: US 2014/0147427 A1
(43) Pub. Date: May 29, 2014

(54) Spray-Dried Lactobacillus Stems/Cells and the Use of Same Against Helicobacter Pylori

(71) Applicants: Stefanie Arya, Berlin (DE); Detlef Goelling, Hattstedt (DE); Caterina Holz, Berlin (DE); Christine Lang, Berlin (DE)

(72) Inventors: Stefanie Arya, Berlin (DE); Detlef Goelling, Hattstedt (DE); Caterina Holz, Berlin (DE); Christine Lang, Berlin (DE)

(73) Assignee: OrganoBalance GmbH, Berlin (DE)

(21) Appl. No.: 14/124,535

(22) PCT Filed: Jun. 8, 2012
(86) PCT No.: PCT/EP2012/060948

§ 371 Date: Feb. 11, 2014

(30) Foreign Application Priority Data
Jun. 8, 2011 (EP) 11169137.4
Oct. 19, 2011 (EP) 11185851.0

(51) Int. Cl. A61K 35/74

(52) U.S. Cl. CPC A61K 35/747
USPC 424/93.45; 435/252.9

(57) ABSTRACT

The invention relates to spray-dried Lactobacillus strains and/or Lactobacillus cells (lactic acid bacteria) and to the uses thereof, in particular for pharmaceutical and/or dietary compositions for the treatment and prophylaxis of Helicobacter pylori infections in humans and animals.

The invention relates to spray-dried Lactobacillus strains and/or Lactobacillus cells (lactic acid bacteria) and to the uses thereof, in particular for pharmaceutical and/or dietary compositions, to include a pharmaceutical product or dietary supplement, for the treatment and prophylaxis of Helicobacter pylori infections in humans and animals.

Probiotic microorganisms include cells which exhibit advantageous effects in human or animal bodies. Probiotic compositions contain such microorganisms. Advantageous effects can be in particular the improvement of the microflora of the digestive tract. In particular, undesired other microorganisms can be inhibited in the microflora by direct interactions between the probiotic microorganisms and the undesired microorganisms, by direct interactions due to inhibition of the metabolism of the undesired microorganism by expression products of the probiotic microorganisms, or by a strengthening of the natural immune system. It is generally assumed that a main mechanism as an essential effective element is the competitive colonization of the gastrointestinal tract, whereby undesired microorganisms can no longer colonize the mucous membrane (mucosa) to an interfering extent or are displaced.

One group of probiotic microorganisms is formed by Lactobacillus strains, for example. These are typically Gram-positive, microaerophilic or anaerobic bacteria, which ferment sugar, forming acids, notably lactic acid.

A pharmaceutical composition containing Lactobacilli, among other things, is known from U.S. Pat. No. 5,716,615. One of the uses of this composition is the treatment of conditions of the gastrointestinal tract.

Lactobacillus strains are known from WO 2004/087891, which are suitable for the production of pharmaceutical or dietary compositions for treating infections of the gastrointestinal tract with Helicobacter pylori.

Interactions of Lactobacilli with Helicobacter pylori are described, among other things, in Wang et al., Am. J. Clin. Nutr. 80:737-41 (2004), Felley et al., Best Practice & Research Clinical Gastroenterology 17 (5): 785-791 (2003), Cazzato et al., Scandinavian Journal of Nutrition 48(1): 26-31 (2004) and Sgouras et al., Applied and Environmental Microbiology 70 (1): 518-526 (2004).

Helicobacter pylori is a spiral-shaped bacterium colonizing the stomach, wherein the pH value in the stomach is increased by the production of urease, thus protecting the bacteria from the acid in the stomach. The bacteria penetrate the mucous membrane and deposit on the epithelial cells. Such an infection activates the body's own immune system, however the immune response is not sufficiently effective to eliminate the infection, resulting in an intensifying immune response, which leads to chronic inflammation and disease, such as gastritis or gastric ulcers, and ultimately to cancer.

When cells gather among each other and form agglomerates, this process is referred to as aggregation. When only one type of cell is involved in this aggregate formation, this is referred to as auto-aggregation or self-aggregation. If at least two different types of cells are involved in the aggregate formation, this process is referred to as co-aggregation. WO 2007/073709 by the applicant describes co-aggregates of Lactobacilli with Helicobacter pylori, which can be utilized for the prophylaxis, treatment and/or eradication therapy of Helicobacter pylori infections; in particular at least a reduction in Helicobacter pylori is achieved. Moreover, Lactobacillus strains that are suited for this purpose are described in WO 2007/073709, which have been filed at the Deutsche Sammlung von Mikroorganismen and Zellkulturen GmbH (DSMZ), Mascheroder Weg 1b, D-39124 Braunschweig, Germany, notably: DSM 17646, DSM 17647, DSM 17648, DSM 17649, DSM 17650, DSM 17651, DSM 17652 and DSM 17653.

The suitable Lactobacillus cells form co-aggregates upon contact with Helicobacter pylori cells. The formation of co-aggregates prevents Helicobacter pylori from penetrating into the stomach lining. The Helicobacter pylori cells, notably the cell surfaces thereof, are masked by the Lactobacillus cells, so that the Helicobacter pylori cells are no longer able to bind to the gastric epithelial cells. Inflammatory reactions are avoided as a result of the prevented binding of Helicobacter pylori to gastric epithelial cells. The masked, and thus inactivated, Helicobacter pylori cells are channeled through the gastrointestinal tract in the form of co-aggregates and excreted. It is thus possible to reduce or eradicate the Helicobacter pylori cells in the stomach by administering suitable Lactobacillus cells. The use of Lactobacillus cells can take place prophylactically or curatively. However, this therapeutic approach is in need of improvement.

Therefore, it is the object of the invention to provide an improved method for the prophylaxis and the treatment of Helicobacter pylori infections. In particular such a method is to be provided, which allows improved co-aggregate formation, wherein the colonization of the stomach lining by Helicobacter pylori is considerably better inhibited/reduced by way of co-aggregates that are formed.

The use and the production of spray-dried Lactobacilli are known in the prior art, for example from Gardiner et al., Comparative Survival Rates of Human-Derived Probiotic Lactobacillus paracasei and L. salivarius Strains during Heat Treatment and Spray Drying, Applied and Enviromental Microbiology 66 (6): 2605-2612 (2000) and Teixeira et al., Survival of Lactobacillus delbrueckii ssp. Bulgaricus Following Spray-Drying, J. Dairy Sci 78:1025-1031 (1995) and Tos t al., Spray Drying, Freeze Drying, or Freezing of three different lactic acid bacteria species, Journal of Food Sci., Wiley-Blackwell Publ., Inc. (US).

However, what is not described is the use of spray-dried Lactobacilli for forming co-aggregates with Helicobacter pylori and the particular suitability thereof in the form of a pharmaceutical or dietary composition.

Surprisingly, improved prevention and prophylaxis of Helicobacter pylori infections can be achieved when spray-dried Lactobacillus cells are used for co-aggregation.

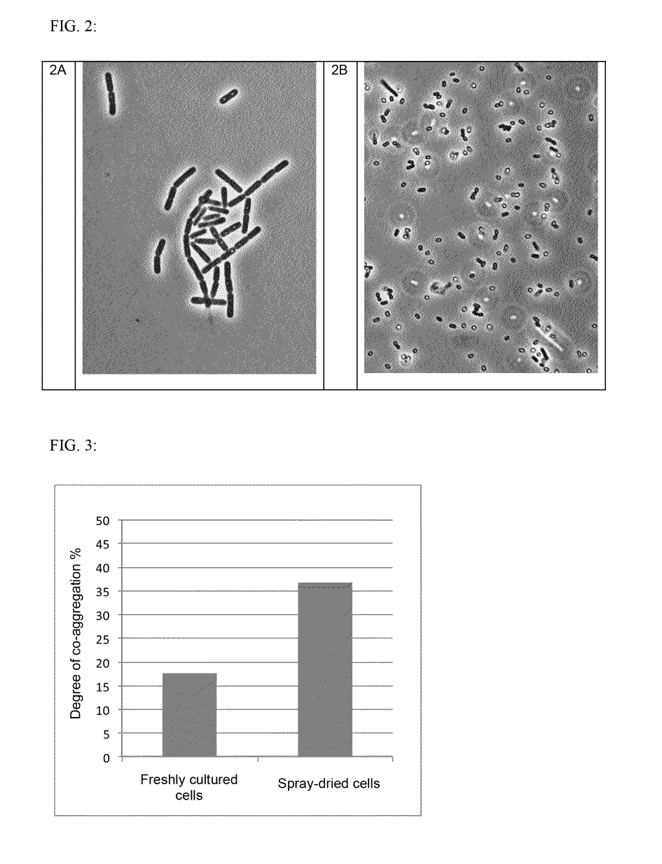

It is particularly advantageous that spray-dried Lactobacillus cells can form large co-aggregates with Helicobacter pylori and, in this way, bring about an efficient reduction in the microbial load of Helicobacter pylori. The spray drying of Lactobacillus cells particularly advantageously results in a decrease in the size of the Lactobacillus cells, wherein additional singulation/separation of the cells takes place. Contrary to other drying methods, or methods carried out without drying, no chains comprising two to ten Lactobacillus cells are obtained (FIG. 2A), but individual cells (mono) or cells of two (dimer) (FIG. 2B)—as is characteristic of spray drying. These spray-dried “germ cells” are excellently suited for the in-situ formation of the co-aggregates after application in the gastric medium, and advantageous, highly dense, compact and efficient co-aggregates composed of Lactobacillus cells and Helicobacter pylori (cells) are obtained, which additionally have advantageous low steric hindrance during the forming phase of the co-aggregates. Such spray-dried Lactobacillus cells according to the invention have a higher binding affinity for Helicobacter pylori than, for example, those that can be produced according to the technical teaching found in WO 2007/073709. As a result of the higher binding affinity, a greater number of Helicobacter pylori cells can be masked and bound by fewer Lactobacillus cells. The higher binding affinity further results in greater stability of the co-aggregates that are formed.

It is further advantageous that spray-dried Lactobacillus cells likewise include a higher proportion of non-live and/or fragments of Lactobacillus cells, which likewise support the spontaneous formation of co-aggregates. The proportion of non-live and/or fragments of Lactobacillus cells can be more than 80%, more than 90%, and even 100%.

The co-aggregates according to the invention pass through the gastrointestinal tract and leave the body naturally. Even if an infection has already occurred, this mechanism of action of spray-dried Lactobacillus strains according to the invention is helpful because further infection with additional Helicobacter pylori bacteria is prevented, and the existing infection can be combated more easily by inactivation/excretion of the Helicobacter pylori bacteria that are present. Additionally, Lactobacillus strains according to the invention are presumably also able to inhibit the urease activity of Helicobacter pylori, so that the Helicobacter pylori bacteria in the co-aggregates lose the protection thereof against the attack of acid in the stomach. Insofar, a synergistic effect is also achieved.

Spray-dried Lactobacillus strains or Lactobacillus cells have the particular advantage that the binding affinity for Helicobacter pylori is increased (supra). Because the surface of Helicobacter pylori is masked by the Lactobacillus cells, Helicobacter pylori cells are prevented from penetrating into the mucous membrane, and consequently a Helicobacter pylori infection, including the chronic inflammation processes, cannot occur and secondary diseases such as gastritis, gastric ulcers or even stomach cancer are effectively prevented.

Moreover, the co-aggregates according to the invention comprising spray-dried Lactobacillus cells and Helicobacter pylori cells exhibit increased stability, so that an increased number of Helicobacter pylori can be incorporated into these co-aggregates, and additionally no renewed release of previously bound Helicobacter pylori cells, for example due to movements of the stomach, takes place. This advantage allows an advantageous lower dosage as compared to the co-aggregates known in the prior art. The spray-dried Lactobacillus cells according to the invention further allow an advantageous increased solubility in water, so that an improved distribution of the spray-dried Lactobacillus cells and of the co-aggregates that are obtained can be achieved in the stomach cavity.

The inventors also found that the spray drying of Lactobacillus cells prevents uncontrolled aggregate formation of the Lactobacilli themselves (auto-aggregation), or at least it is drastically reduced compared to fresh non-spray-dried Lactobacilli. The binding sites for masking Helicobacter pylori are occupied by the auto-aggregation of the Lactobacillus cells among each other. Reducing or preventing auto-aggregation thus likewise results in an advantageous lower dosage of the Lactobacilli that are used as compared to the prior art.

The spraying method according to the invention brings about a morphological change of the Lactobacilli, so that the binding affinity for Helicobacter pylori is increased, and thus improved co-aggregate formation can take place.

The invention thus relates to a pharmaceutical or dietary composition comprising spray-dried Lactobacillus cells for the prophylaxis and the treatment of Helicobacter pylori infections in humans and animals, in particular mammals.

In a further embodiment of the invention, the spray-dried Lactobacillus cells are preferably and essentially present in the composition in a monomer and/or dimer form (supra, FIG. 2B).

The invention thus further relates to such a composition according to the invention in which a.) Lactobacillus cells are spray-dried, and b.) spray-dried Lactobacillus cells that are obtained are introduced into a physiologically compatible carrier (exemplary embodiments of physiologically compatible carriers are described below).

The invention further relates to such a composition, in which, after the application in humans or animals, co-aggregates with Helicobacter pylori are formed in-situ in the gastric medium which are preferably larger, and not smaller, than 50 μm, and in particular larger, and not smaller, than 100 μm, 150 μm, in particular larger than 500 μm, particularly preferably larger than 1,000 μm or 1,100 μm.

The term “Lactobacillus cells” within the meaning of the invention (in the broader sense lactic acid bacteria, also Lactobacilli) includes those microorganisms which require carbohydrates, in particular glucose and lactose, for the fermentation of lactic acid, and primarily employ the Embden-Meyerhof pathway for biosynthesis. In terms of taxonomy, the Lactobacillus cells fall under the family Lactobacteriaceae. They are Gram-positive, not spore-forming, and generally immotile. The Lactobacillus cells live anaerobically, however they are aerotolerant, even though they do not contain any hemins (cytochrome, catalase) (Schleifer et al., System. Appl. Microb.: 18, 461-467 (1995) or Ludwig et al., System. Appl. Microb. 15: 487-501 (1992)). The Lactobacillus cells or the species can be determined based on the carbohydrate fermentation pattern, in particular by way of the API test (from biomerieux company). According to the invention, this includes in particular those species which are suitable for homofermentative lactic acid fermentation or heterofermentative lactic acid fermentation. Further preferred are those Lactobacillus cells which are selected from the group consisting of Lactobacillus lactis, Lactobacillus helveticus, Lactobacillus jensenii, Lactobacillus acidophilus, Lactobacillus bulgaricus, Lactobacillus amylovorus, Lactobacillus delbrueckii, Lactobacillus casei, Lactobacillus crispatus, Lactobacillus gasseri, Lactobacillus johnsonii, Lactobacillus paracasei, Lactobacillus pentosus, Lactobacillus rhamnosus, Lactobacillus curvatus and Lactobacillus plantarum (all homofermentative), further Lactobacillus brevis, Lactobacillus buchneri, Lactobacillus fructivorans, Lactobacillus hilgardii, Lactobacillus fermentum, Lactobacillus reuteri, Lactobacillus viridescens and Bifidobacterium bifidum (all heterofermentative). Exemplary suitable Lactobacillus cells from the applicant are filed: DSM 17646, DSM 17647, DSM 17648, DSM 17649, DSM 17650, DSM 17651, DSM 17652 and DSM 17653 (supra). Lactobacillus fermentum, Lactobacillus brevis, Lactobacillus reuteri, Lactobacillus buchneri and Lactobacillus pentosus are preferred according to the invention.

Also covered according to the invention are those “Lactobacillus cells”, including “derivatives” or “analogs” or “mutants” thereof, which are inactivated, live or non-live, or represent parts and fragments, for example enzymatic or mechanical decomposition products (for example French Press and the like), of Lactobacillus cells, to the extent these are suitable for co-aggregation.

The terms “derivatives”, “analogs”, “mutants” or “inactivated” in the present case also include cell or fermentation supernatants, lysates, fractions or extracts of the “Lactobacillus cells”, wherein these cell or fermentation supernatants, lysates, fractions or extracts preferably have the properties of the Lactobacillus cells/strains/microorganisms according to the invention. To this end, “lysate”—as well as the term “extract”—in particular denotes a solution or suspension in an aqueous medium of the inventive cells of the microorganism and comprises macromolecules, for example, such as DNA, RNA, proteins, peptides, lipids, carbohydrates and the like, and notably also cell debris. The lysate preferably also comprises the cell wall or cell wall constituents. Methods for producing lysates are sufficiently known to a person skilled in the art and include, for example, the use of a “French press” or enzymatic lysis, a ball mill with glass beads or iron spheres. The cells can be disrupted enzymatically, physically or also chemically. Examples of enzymatic cell lysis can be individual enzymes as well as enzyme cocktails, such as proteases, Proteinase K, lipases, glycosidases; chemical lysis can be effected by ionophores, detergents such as SDS, acids or bases; physical methods can be brought about by high pressures such as a French press, osmolarities, temperatures, or alternating between hot and cold conditions. Moreover, it is of course possible to combine chemical, physical and enzymatic methods.

“Derivatives” or “analogs” or “mutants” or “inactivated” of the Lactobacillus cells/strains/microorganisms according to the invention preferably have the same properties as the cited strains. To this end, metabolic activity preferably no longer exists with the “inactivated (form)” or “derivative” or “analog”.

“Analogs” of the Lactobacillus cells/strains/microorganisms according to the invention constitute a form of the lysate or fragments. A “fragment” of the Lactobacillus cells/strains/microorganisms according to the invention constitutes a part of the cells, for example the cell membrane, macromolecules, such as DNA, RNA, proteins, peptides, lipids, carbohydrates and the like, as well as cell debris. A person skilled in the art will be able to assign the proper content to the terms “analogs”, “fragments”, “derivatives” or “mutants” and interpret the terms within the meaning of the present invention. So as to provide mutants, derivatives, fragments or analogs of the preferred Lactobacillus cells/strains/microorganisms, a person skilled in the art can resort to the standard literature that is available and discloses techniques which can be employed to produce mutants, derivatives, fragments or analogs. Mutants or genetically modified variants or derivatives are genetically modified, for example by way of recombinant DNA technologies (cloning, sequencing, transformation of recombinant nucleic acids), as well as physical mutagenesis, for example by way of ultraviolet radiation, but also by chemical agents, such as ethyl methanesulfonate (EMS). For this purpose, changes in the positive properties can be selected—either in a targeted manner or by evaluating a plurality of mutants that resulted. Genetically modified mutants include cells of the microorganisms according to the invention and house recombinant nucleic acids in the bacterial chromosome and/or plasmid thereof Modifications by way of point mutations can additionally cause effects on the expression/transcription/translation, as do spontaneous mutations without direct genetic manipulation (for example, see J. Sambrook, E. F. Fritsch, T. Maniatis, Cold Molecular cloning: a laboratory manual/Spring Harbor Laboratory Press, 3rd edition (2001)).

All these microorganisms are referred to as “Lactobacillus cells” above and hereafter.

The essential culture conditions of the human gastric tract include a pH value in the range of 1.8 to 4.5 and the presence of pepsin as well as NaCl. A reference medium that is characteristic of such culture conditions comprises the following components: water, 5 g/l NaCl and 3 g/l pepsin, wherein the pH value is adjusted to 2.0 or 4.0 with hydrochloric acid so as to simulate an empty or full stomach.

The term of “co-aggregation” within the meaning of the invention denotes the formation of cell aggregates having a size of at least 50 μm or 100 μm and more, containing spray-dried Lactobacillus cells according to the invention and Helicobacter pylori cells, in suspensions, for example according to the following examples, in particular in a reference medium, as described above.

The term “spray-dried Lactobacillus cells” within the meaning of the invention denotes that the Lactobacillus cells are dried using a spray drying or atomizing method (synonymous), wherein a suspension of Lactobacillus cells is dispersed into fine mist-like droplets, for example, and a powder can be obtained.

During spray drying according to the invention, a solution or suspension containing Lactobacillus cells is sprayed into a hot drying medium, whereby it is dried. The mixture to be sprayed can be present in the form of a solution, an emulsion, a suspension or dispersion. It is atomized into millions of individual droplets with the aid of a nozzle or a spraying wheel, drastically increasing the surface. The solvent, such as water, is immediately evaporated by the hot air and is discharged. Moreover, the Lactobacillus cells are spray-dried alone.

The spray drying or atomization method can be distinguished from other drying methods since the use of a nozzle or similarly acting means is required, such as a unary nozzle, hollow cone nozzle, pressure nozzle, binary nozzle externally mixing, pneumatic nozzle, binary nozzle internally mixing, atomizing disk or ultrasonic atomizer.

Spray drying methods are described in the prior art and are familiar to the person skilled in the art (see Gardiner et al., Teixeira et al. (supra) or EP74050 and EP285682). Devices are known and described as relevant, such as the mini spray dryer B-191 or B-290 by Büchi Labortechnik AG (Germany) or SD-6.3-R by GEA Niro (Denmark). It is further known that arbitrary adjuvants and additives can be used.

The invention further relates to a pharmaceutical and/or dietary composition comprising a physiologically effective dose of spray-dried Lactobacillus cells according to the invention and a physiologically compatible carrier. The pharmaceutical compositions are compositions which serve solely therapeutic or prophylactic purposes, wherein, in addition to Lactobacillus cells, only adjuvants and/or excipients that are common in galenics are present. The dietary compositions within the meaning of the present invention are composition which, in addition to the spray-dried Lactobacillus cells according to the invention, comprise a food or foodstuff (see, for example, not exhaustively, EU Directive 2002/46/EC of Jun. 10, 2002) and/or a feedstuff for pets and/or farm animals and/or dietary supplement, optionally comprising adjuvants and additives.

The invention further relates to the use or application of spray-dried Lactobacillus cells according to the invention for producing a pharmaceutical or dietary composition, or a pharmaceutical product or a dietary supplement, comprising spray-dried Lactobacillus cells or a pharmaceutical or dietary composition, in particular for the prophylaxis and/or the treatment of diseases caused by Helicobacter pylori infections, for example gastrointestinal conditions. These include in particular gastritis, stomach ulcers and stomach cancer. Also covered are those diseases and symptoms such as abdominal discomfort, in particular discomfort of the upper abdomen, gastric heaviness, gastric spasms, stomach pain, pain or pressure in the upper abdomen, burning sensation in the upper abdomen, chronic-recurrent abdominal disorders, permanent feeling of fullness, lack of appetite, fasting pain, bloating, heartburn, diarrhea, irregular bowel movements, indisposition, nausea, sickness and vomiting, food intolerance, malabsorption, upset stomach (functional dyspepsia), gastritis, damage to the mucous membrane, or gastroduodenal ulcer.

The invention further relates to the use or application of the spray-dried Lactobacillus cells according to the invention, or a pharmaceutical product or a dietary supplement comprising spray-dried Lactobacillus cells or a pharmaceutical or dietary composition, for eradicating or for the eradication therapy of Helicobacter pylori, optionally in combination with further suitable active ingredients, such as antibiotics.

A pharmaceutical or dietary composition, in particular a dietary supplement or pharmaceutical product (drug), according to the invention may be characterized by comprising 10² to 10¹⁵, preferably 10⁴ or 10⁸ to 10¹², in particular 10⁸ to 10¹⁰, spray-dried Lactobacillus cells. The reference quantity here is a unit of administration, for example a tablet. The composition is preferably prepared for oral administration.

The galenic preparation of a pharmaceutical or dietary composition according to the invention, in particular of a dietary supplement or pharmaceutical product (drug), can be carried out in a way that is common practice in the art. Suitable solid or liquid galenic forms of preparation include, for example, granules, powders, sugar-coated tablets, tablets (micro)capsules, hard capsules, suppositories, syrups, juices, suspensions or emulsions, in the production of which conventional adjuvants such as excipients, disintegrants, binders, coating agents, swelling agents, glidants, lubricants, flavor additives, sweetening agents and solubilizers, are employed. Adjuvants that should be mentioned include magnesium stearate, sodium chloride, magnesium carbonate, titanium dioxide, lactose, mannitol and other sugars, talcum, milk protein, gelatin, starch, cellulose and the derivatives thereof, animal and vegetable oils such as cod liver oil, sunflower oil, peanut oil or sesame oil, polyethylene glycols and solvents, such as sterile water, and monohydric or polyhydric alcohols, such as glycerin. A pharmaceutical composition according to the invention can be produced by mixing cells of at least one Lactobacillus strain that is used according to the invention in a defined dosage with a pharmaceutically suitable and physiologically compatible carrier, and optionally with further suitable active ingredients, additives or adjuvants having defined dosages, and preparing the desired form of administration. Possible carriers include in particular substances that are selected from the group consisting of maltodextrin, microcrystalline cellulose, starch, in particular corn starch, levulose, lactose, dextrose, and mixtures of such substances. The composition may comprise or consist of 0.1 to 95% by weight carrier and 5 to 99.9% by weight spray-dried Lactobacillus cells, based on the total quantity of cells and carrier.

In the case of the dietary composition and/or dietary supplement, it may be provided that the composition comprises 10² to 10¹⁵, preferably 10⁴ to 10¹², and more particularly 10⁸ to 10¹⁰ Lactobacillus cells. The reference quantity is a unit of administration, for example a packaging unit of a foodstuff for sale to an end consumer. The physiologically compatible carrier will generally be a food, which in particular is selected from the group consisting of dairy products, fermented dairy products, milk, yogurt, cheese, cereals, granola bars, baked goods, beverages and infant food preparations. Suitable foods or foodstuffs, including water, according to the invention are those as defined, not exhaustively, for example, in (EC) Regulation No. 178/2002 of Jan. 28, 2002.

The invention further relates a method for producing a pharmaceutical and/or dietary composition according to the invention, in particular a dietary supplement or pharmaceutical product (drug), wherein the spray-dried Lactobacillus cells are mixed with the physiologically compatible carrier and are preferably prepared for oral administration.

The invention moreover relates to a method for producing a composition according to the invention, wherein the Lactobacillus cells are (i) optionally inoculated and enriched, (ii) fermented, and (iii) spray-dried, and subsequently mixed with a physiologically compatible carrier and are preferably prepared for oral administration.

Finally, the invention relates to a method for the prophylaxis or the treatment of a human or an animal, in particular a patient or test subject suffering, or at risk of suffering, from a condition caused by a Helicobacter pylori infection, in particular gastritis or stomach ulcer, wherein this person is administered a physiologically effective dosage of a pharmaceutical and/or dietary composition according to the invention once to five times a day. The administration can take place over a limited period of time, for example 1 to 30 weeks, or without limitations in terms of time. In particular the latter is suited for permanent prophylaxis and for the prevention of relapses.

The invention will be described in greater detail hereafter based on examples that represent only embodiments, without intending to limit the invention to these examples.

### EXAMPLES AND FIGURES

### Example 1

### Storage of Strains Used

The Lactobacillus strains were stored in the frozen state. 1 ml of a culture cultured up to the stationary phase (OD₆₀₀/ml 4-8) in MRS medium (55 g/l, pH 6.5; Difco, USA) was mixed with 500 μl of a 50% (v/v) sterile glycerin solution, and the mixture was frozen at −80° C.

Helicobacter pylori was stored in the frozen state. 1 ml of a culture cultured up to the stationary phase in Brucella broth (28 g/l, pH 7.0; BD, USA), supplemented with 10% (v/v) fetal calf serum (Biochrom), was mixed with 500 μl of a 50% (v/v) sterile glycerin solution, and the mixture was frozen at −80° C.

### Example 2

### Process for Producing Spray-Dried Lactobacillus Cells

A spray-dried Lactobacillus, which is able to co-aggregate with Helicobacter pylori under culture conditions of the human digestive tract, in particular the stomach, was produced as described below:

The starter culture 1 took place in a 15 ml reaction vessel in 10 ml MRS medium. The medium was inoculated with freshly cultured, deep-frozen, freeze-dried or spray-dried cells of the Lactobacillus strain. This starter culture 1 was incubated for 24 hours at 37° C. under anaerobic conditions. The entire starter culture 1 was used to inoculate starter culture 2. Starter culture 2 was composed of 240 ml MRS medium with 10 ml of starter culture 1. Starter culture 2 was incubated for 19 hours at 37° C. under anaerobic conditions. At the end of each starter culture, the optical density, pH value and cfu were determined.

5 liters of MRS were used for the fermentation of Lactobacillus. The fermenter, including all the necessary components, was autoclaved. Desired values for the temperature and stirrer speed were 37° C. and 150 rpm, respectively. The inoculation concentration was 5%. At the end of the fermentation process, the cell count, colony-forming units, pH, optical density at 600 nm, and the concentration of glucose and lactate were determined Thereafter, the fermentation broth was concentrated ten fold to twenty fold Thereafter, the concentrate was frozen.

Prior to drying, the twenty-fold concentrate was thawed and centrifuged for 15 minutes at 4300×g. The cells were then washed once with 0.9% NaCl solution and again centrifuged for 15 minutes at 4300×g. Thereafter, the cells were resuspended in 500 ml 10% NaCl solution.

The spray drying process was carried out in a Büchi Mini Spray Dryer B-191. The inlet temperature was 140° C. The outlet temperature was 86° C. The hot air flow was 500 L/h. The aspirator power was 75%, the pumping rate was 5%. The cell suspension was dried using the above-described parameters, and thereafter the spray-dried Lactobacillus powder was removed.

The total cell count as well as the viable cell count of the Lactobacillus strain per gram of spray-dried powder were the determined The microscopic examination of the spray-dried powder (FIG. 2B) showed that the Lactobacillus cells are smaller than non-spray-dried Lactobacillus cells (FIG. 2A), and that the spray-dried Lactobacillus cells are present in monomer and dimer forms, while the non-spray-dried Lactobacillus cells have longer chains. The spray-dried Lactobacillus cells were stored at 4° C. until further use.

### Example 3

### Comparison of the Co-Aggregation of Helicobacter pylori by Non-Spray-Dried and Spray-Dried Lactobacillus Strains or Lactobacillus Cells Under Stomach Conditions

The Lactobacillus cells were cultured in closed 15 ml tubes in MRS medium at 37° C. for 24 hours. The spray-dried powder was resuspended in PBS having a concentration of 10 mg/ml for the examination of spray-dried Lactobacilli. Helicobacter pylori was cultured for approximately 2 days in Erlenmeyer flasks under microaerophilic conditions in Brucella broth (28 g/l, pH 7.0; BD, USA) with 10% fetal calf serum (Biochrom) at 37° C. After culturing, the cell morphology of Helicobacter pylori was microscopically analyzed. Assays were carried out with cells having sigmoidal morphology or with cells having coccoid morphology. Cultures having mixed morphology were also examined

The respective cells were harvested by way of centrifugation at 3200 g for 10 minutes, and the supernatant was discarded. The Lactobacillus cells were washed once in 5 ml buffer and resuspended in 5 ml PBS buffer (PBS buffer containing 1.5 g/l Na₂HPO₄*2H₂O, 0.2 g/l KH₂PO₄ and 8.8 g/l NaCl). The Helicobacter pylori cells were washed once in 5 ml PBS buffer and resuspended in 5 ml artificial gastric juice (containing 5 g/l NaCl and 3 g/l pepsin (Sigma)). The OD₆₀₀ value was measured for the respective cells and adjusted to a value of 2 for Helicobacter pylori and 4 for Lactobacillus by adding artificial gastric juice and PBS buffer, respectively.

2.5 ml of each cell suspension thus obtained (Helicobacter pylori/Lactobacillus) was mixed, and the mixture was shaken for 10 seconds to 10 minutes. The result was optically visible due to considerable flocculation in the samples that contained Helicobacter pylori and Lactobacillus and was also microscopically analyzed.

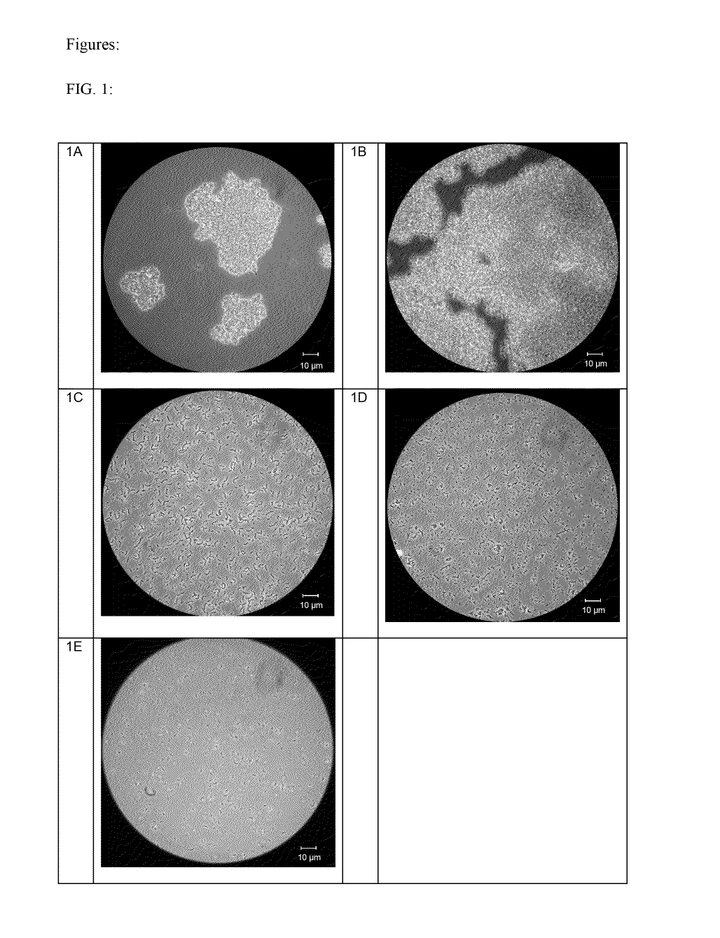

FIG. 1A shows the microscopic image of co-aggregates comprising Helicobacter pylori and non-spray-dried Lactobacillus DSM 17648 cells. FIG. 1B shows the microscopic image of co-aggregates comprising Helicobacter pylori and spray-dried Lactobacillus DSM 17648 cells. These co-aggregates are considerably larger than the co-aggregates comprising non-spray-dried Lactobacillus cells. Control experiments regarding self-aggregation were carried out by separately analyzing respective cultures comprising Lactobacillus and Helicobacter pylori alone. Neither co-aggregation nor auto-aggregation is apparent in FIGS. 1C, 1D and 1E. FIG. 1C shows the microscopic image of freshly cultured cells of Lactobacillus DSM 17648 under artificial stomach conditions. FIG. 1D shows the microscopic image of spray-dried cells of Lactobacillus DSM 17648 under artificial stomach conditions. FIG. 1E shows the microscopic image of freshly cultured Helicobacter pylori cells under artificial stomach conditions.

### Example 4

### Testing the Stability of the Co-Aggregates Between Spray-Dried Lactobacillus Cells and Helicobacter pylori Cells

The experiments as in Example 3 were carried out to test the stability of the co-aggregates under in vivo conditions. Co-aggregates between Lactobacillus cells and Helicobacter pylori cells that formed after 5 minutes of shaking were exposed to strong shearing forces by pipetting the suspension for 1 minute or by shaking for 2 minutes at high speed. Thereafter, the co-aggregate size was analyzed microscopically and macroscopically. The sizes of the co-aggregates comprising spray-dried Lactobacillus cells and Helicobacter pylori did not decrease, however the sizes of the co-aggregates of non-spray-dried Lactobacillus cells and Helicobacter pylori decreased.

### Example 5

### Production of a Pharmaceutical Composition Comprising Lactobacillus Strains According to the Invention

Cells of a Lactobacillus strain or of multiple Lactobacillus strains of the invention are pulled according to Examples 1 and 2 and spray-dried. The spray-dried powder is then ground to a particle size of no more than approximately 1 mm in diameter. The granules obtained are mixed with excipients and/or adjuvants using the following proportions (% by weight): 20% granules, 2% silicon dioxide (Syloid AL-IFP, GRACE Davidson), 1% magnesium stearate (MF-2-V, Ackros), 77% microcrystalline cellulose (Avicel PH 112, FMC).

Mixing is carried out in a Quintech Micromixer at Position 70 Level II. All components are added simultaneously. Mixing is carried out for approximately 120 seconds. Thereafter, the mixture obtained is pressed using a commercially available tablet press under customary conditions, however using a low pressing force (<10 kN) to form tablets having a weight of approximately 500 mg. Each tablet contains approximately 10⁸ to 10¹⁰ spray-dried Lactobacillus cells.

### Example 6

### Comparison of the Co-Aggregation of Helicobacter pylori by Non-Spray-Dried and Spray-Dried Lactobacillus Strains or Lactobacillus Cells Using Various Lactobacillus Cell Counts

The Lactobacillus cells were cultured in closed 15 ml tubes in MRS medium at 37° C. for 24 hours. The spray-dried powder was resuspended in PBS having a concentration of 10 mg/ml for the examination of spray-dried Lactobacillus cells. Helicobacter pylori was cultured for approximately 2 days in Erlenmeyer flasks under microaerophilic conditions in Brucella broth (28 g/l, pH 7.0; BD, USA) with 10% fetal calf serum (Biochrom) at 37° C. After culturing, the cell morphology of Helicobacter pylori was microscopically analyzed. Assays were carried out with cells having sigmoidal morphology or with cells having coccoid morphology. Cultures having mixed morphology were also examined

The respective cells were harvested by way of centrifugation at 3,200 g for 10 minutes, and the supernatant was discarded. The Lactobacillus cells were washed once in 5 ml buffer and resuspended in 5 ml PBS buffer (PBS buffer containing 1.5 g/l Na₂HPO₄*2H₂O, 0.2 g/l KH₂PO₄ and 8.8 g/l NaCl). The Helicobacter pylori cells were washed once in 5 ml PBS buffer and resuspended in 5 ml artificial gastric juice (containing 5 g/l NaCl and 3 g/l pepsin (Sigma)). The OD600 value was measured for the respective cells and adjusted to a value of 2 for Helicobacter pylori and 4 for Lactobacillus by adding artificial gastric juice and PBS buffer, respectively.

A cell count determination was carried out for the Lactobacillus suspensions by way of a Thoma counting chamber, and dilutions were produced so as to obtain suspensions having differently defined cell counts. 2.5 ml of each cell suspension thus obtained (Helicobacter pylori/Lactobacillus) was mixed, and the mixture was shaken for 5 minutes. After a residence time of 2 minutes, the result was optically visible due to considerable flocculation in the samples that contained Helicobacter pylori and Lactobacillus and was also microscopically visible (pictures are not shown). 1 ml was withdrawn from the co-aggregate-free phase (supernatant; sediment co-aggregates) so as to quantify the co-aggregation and the OD600 was determined Using the formula

$$Degree of co \text{-} aggregation \% = \frac{( {OD}_{H . pylori} + {OD}_{Lactobacillus} ) - {OD}_{H . p . + Lactobacillus}}{{OD}_{H . pylori} + {OD}_{Lactobacillus}} \times 100$$

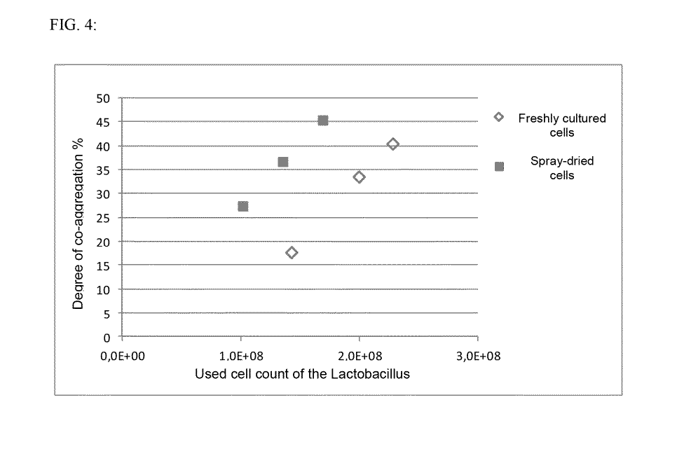

the degree of co-aggregation (in %) was determined. FIGS. 3 and 4 show the degree of co-aggregation of freshly cultured and spray-dried Lactobacillus DSM 17648 cells with respect to Helicobacter pylori. 0% represents the OD600 of the Helicobacter pylori (in the supernatant) without the addition of Lactobacillus cells (no co-aggregation or no sedimentation due to co-aggregation). 100% degree of aggregation represents a maximum degree of co-aggregation, which is to say an OD600 of 0, or the OD600 of the medium used without cells (all Helicobacter pylori cells sediment due to co-aggregation, and no Helicobacter pylori cells can be detected in the supernatant by way of OD measurement).

At identical cell counts, the degree of co-aggregation with Helicobacter pylori of spray-dried Lactobacillus cells is more than twice that of freshly cultured cells (FIGS. 3 and 4).

### DESCRIPTION OF THE FIGURES

FIG. 1A shows co-aggregates comprising freshly cultured, non-spray-dried Lactobacillus DSM 17648 cells and Helicobacter pylori (co-aggregates have a size of 40 μm and more) under simulated stomach conditions.

FIG. 1B shows a very large co-aggregate comprising spray-dried Lactobacillus DSM 17648 cells and Helicobacter pylori (co-aggregates have a size of 150 μm and more) under simulated stomach conditions.

FIGS. 1C to 1E serve as controls in the experiment.

FIG. 1C shows freshly cultured Lactobacillus DSM 17648 cells under simulated stomach conditions. No auto-aggregation can be detected.

FIG. 1D shows spray-dried Lactobacillus DSM 17648 cells under simulated stomach conditions. No auto-aggregation can be detected.

FIG. 1E shows freshly cultured Helicobacter pylori cells under simulated stomach conditions. No auto-aggregation can be detected. (FIGS. 1A-E are 1000-fold enlarged)

FIGS. 2A and 2B show the morphology of the Lactobacillus cells.

FIG. 2A shows non-spray-dried Lactobacillus cells. The cells are present in chains of 2 to 10 cells.

FIG. 2B shows spray-dried Lactobacillus cells. The cells are present in monomer and dimer forms (1 to 2 cells) and are significantly smaller than non-spray-dried cells.

FIG. 3: Comparison of the Degree of Co-Aggregation of Freshly Cultured and Spray-Dried Cells of Lactobacillus DSM 17648 with respect to Helicobacter pylori. In each case, 1.4×10⁸ Lactobacillus cells were used in the tests. At identical cell counts, the degree of co-aggregation activity with Helicobacter pylori of spray-dried Lactobacillus cells is more than twice that of freshly cultured cells. 0% represents the OD600 of the Helicobacter pylori (in the supernatant) without the addition of Lactobacillus cells (no co-aggregation or no sedimentation due to co-aggregation). 100% degree of aggregation represents a maximum degree of co-aggregation, which is to say an OD600 of 0, or the OD600 of the medium used without cells (all Helicobacter pylori cells sediment due to co-aggregation, and no Helicobacter pylori cells can be detected in the supernatant by way of OD measurement).

FIG. 4: Comparison of the Degree of Co-Aggregation of Freshly Cultured and Spray-Dried Cells of Lactobacillus DSM 17648 with respect to Helicobacter pylori. In each case, different quantities of Lactobacillus cells were used in the tests. The degree of co-aggregation activity with Helicobacter pylori of spray-dried Lactobacillus cells is twice that of freshly cultured cells. 0% represents the OD600 of the Helicobacter pylori (in the supernatant) without the addition of Lactobacillus cells (no co-aggregation or no sedimentation due to co-aggregation). 100% degree of aggregation represents a maximum degree of co-aggregation, which is to say an OD600 of 0, or the OD600 of the medium used without cells (all Helicobacter pylori cells sediment due to co-aggregation, and no Helicobacter pylori cells can be detected in the supernatant by way of OD measurement).

1-15. (canceled)

16. A composition comprising spray-dried Lactobacillus cells for use in the treatment and prophylaxis of Helicobacter pylori infections in humans or animals.

17. The composition of claim 16, wherein said spray-dried Lactobacillus cells are essentially present in monomer and/or dimer form.

18. The composition of claim 16, wherein:
a) Lactobacillus cells are spray-dried; and
b) spray-dried Lactobacillus cells that are obtained are introduced into a physiologically compatible carrier.

19. The composition of claim 16, wherein co-aggregates are formed with Helicobacter pylori in situ after application in the stomach medium.

20. The composition of claim 19, wherein the co-aggregates are not smaller than 50 μm, and more particularly are larger than 500 μm.

21. The composition of claim 16, wherein the Lactobacillus cells are selected from the group consisting of Lactobacillus lactis, Lactobacillus helveticus, Lactobacillus jensenii, Lactobacillus acidophilus, Lactobacillus bulgaricus, Lactobacillus amylovorus, Lactobacillus delbrueckii, Lactobacillus casei, Lactobacillus crispatus, Lactobacillus gasseri, Lactobacillus johnsonii, Lactobacillus paracasei, Lactobacillus pentosus, Lactobacillus rhamnosus, Lactobacillus curvatus and Lactobacillus plantarum, Lactobacillus brevis, Lactobacillus buchneri, Lactobacillus fructivorans, Lactobacillus hilgardii, Lactobacillus fermentum, Lactobacillus reuteri, Lactobacillus viridescens, DSM 17646, DSM 17647, DSM 17648, DSM 17649, DSM 17650, DSM 17651, DSM 17652, DSM 17653.

22. The composition of claim 16, wherein the Lactobacillus cells are selected from the group consisting of Lactobacillus fermentum, Lactobacillus brevis, Lactobacillus reuteri, Lactobacillus buchneri and Lactobacillus pentosus.

23. A pharmaceutical or dietary composition comprising the composition of claim 16 and optionally further comprising adjuvants and additives.

24. The pharmaceutical or dietary composition of claim 23, in form of food or foodstuff and/or feedstuff for pets and/or farm animals, or in form of dietary supplement.

25. The composition of claim 16 for eradication therapy of Helicobacter pylori, optionally in combination with further suitable active ingredients.

26. A pharmaceutical product comprising spray-dried Lactobacillus cells or the composition of claim 16 for use with Helicobacter pylori infections in humans or animals.

27. A dietary supplement comprising spray-dried Lactobacillus cells or the composition of claim 16 for use with Helicobacter pylori infections in humans or animals.

28. A method for producing the composition of claim 16, or a pharmaceutical product or dietary supplement comprising said composition, comprising:
(i) fermenting Lactobacillus cells;
(ii) spray-drying the Lactobacillus cells; and
(iii) mixing the spray-dried Lactobacillus cells with a physiologically compatible carrier,
optionally the Lactobacillus cells are first inoculated and enriched.

29. A method for the prophylaxis and/or treatment of conditions caused by an infection with Helicobacter pylori in a human or an animal, comprising administering a pharmaceutical or dietary composition comprising spray-dried Lactobacillus cells to a human or an animal in need thereof.

30. The method of claim 29, wherein said conditions caused by an infection with Helicobacter pylori are selected from the group consisting of gastrointestinal conditions, gastritis, stomach ulcers, stomach cancer, abdominal discomfort, discomfort of the upper abdomen, gastric heaviness, gastric spasms, stomach pain, pain or pressure in the upper abdomen, burning sensation in the upper abdomen, chronic-recurrent abdominal disorders, permanent feeling of fullness, lack of appetite, fasting pain, bloating, heartburn, diarrhea, irregular bowel movements, indisposition, nausea, sickness and vomiting, food intolerance, malabsorption, upset stomach (functional dyspepsia), gastritis, damage to the mucous membrane, and gastroduodenal ulcer.

31. An eradication therapy of Helicobacter pylori comprising utilizing spray-dried Lactobacillus cells or the composition of claim 16, optionally in combination with further suitable active ingredients.

32. The eradication therapy of claim 31, wherein said suitable active ingredients are antibiotics.

33. A pharmaceutical product or dietary supplement for the prophylaxis and/or treatment of conditions caused by infections with Helicobacter pylori, or for use in the eradication therapy of Helicobacter pylori, comprising spray-dried Lactobacillus cells or the composition of claim 16, optionally in combination with further suitable active ingredients.

34. The pharmaceutical product or dietary of claim 33, wherein the conditions caused by infections with Helicobacter pylori are selected from the group consisting of gastrointestinal conditions, gastritis, stomach ulcers, stomach cancer, abdominal discomfort, discomfort of the upper abdomen, gastric heaviness, gastric spasms, stomach pain, pain or pressure in the upper abdomen, burning sensation in the upper abdomen, chronic-recurrent abdominal disorders, permanent feeling of fullness, lack of appetite, fasting pain, bloating, heartburn, diarrhea, irregular bowel movements, indisposition, nausea, sickness and vomiting, food intolerance, malabsorption, upset stomach (functional dyspepsia), gastritis, damage to the mucous membrane, and gastroduodenal ulcer.

35. The pharmaceutical product or dietary of claim 33, wherein suitable active ingredients are antibiotics.
